(19) United States
(12) Patent Application Publication
(10) Pub. No.: US 2009/0327102 A1
(43) Pub. Date: Dec. 31, 2009

(54) SYSTEM AND METHOD FOR PROVIDING REAL TIME ASSET VISIBILITY

(76) Inventors: Jatin Maniar, Sunnyvale, CA (US); Hitesh Kalra, Pleasant Hill, CA (US)

Correspondence Address: EVERGREEN VALLEY LAW GROUP, 4 North Second Street, Suite 598, SAN JOSE, CA, 95113, US

(21) Appl. No.: 11/690,131

(22) Filed: Mar. 23, 2007

(51) Int. Cl. G06Q 10/00

(52) U.S. Cl. 705/28; 235/385

(57) ABSTRACT

A method and system for providing real time asset visibility for a plurality of assets and for providing a set of applications based on real time asset visibility, in particular, for the health care facility. The method includes the steps of collecting tag data from a plurality of active readers, identifying a particular asset coupled to the tag data and identifying the zone where the particular asset is located using an algorithm; and associating an asset state information, tag data, zone information, and other device information in the same environment of the asset for providing real time asset visibility. The invention activates an alerts frame work and sends action messages to the respective departments in the health care facility and also supports a set of applications including searching for availability of assets and location, data mining applications, dashboard applications, handheld applications, mapping applications, and reporting services.

### BACKGROUND

1. Technical Field

The present invention relates generally to asset management and more particularly to providing asset visibility in real time.

2. Discussion of Prior Art

Asset management and “asset visibility” are crucial in reducing operating costs in hospital environments. Some tools are available to track and manage assets automatically. Automatic Identification tags such as Radio Frequency IDentification (RFID) tags can be attached to assets, and RFID readers used to read the tag data to provide asset visibility. In a zonal approach to tracking assets, larger areas require multiple devices, which reduces a system's reliability.

An asset tracking system may include various hardware and communication technologies. Readers associated with different types of tags operate in different frequencies (e.g. 802.11 or Wi-Fi, 433 MHz technology etc) and perform real time asset visibility in different ways. An asset visibility system should coexist with multiple technologies and multiple hardware vendors.

Hospitals need asset visibility at the room level, to indicate, for example, how many clean IV pumps are available in each room. Conventional asset visibility systems provide mere asset visibility and hardware support for the asset visibility system. One prior art system is the asset tracking solution by Aeroscout®, Ltd., which is designed to support only their proprietary hardware.

Similar asset visibility issues exist in other environments where business and productivity rely on the visibility and tracking of mobile assets. Several examples are inventory management, shipping facilities and other facilities which include mobile assets.

Hence, there is a need for a reliable, real time asset visibility and functionality system for enterprise facilities or a plurality such as hospitals, manufacturing or corporate office environment.

### SUMMARY

The present invention teaches a system and method for providing real time asset visibility, and teaches a set of applications based on the real time asset visibility, particularly for health care facilities.

An example method provides real time asset visibility and manages assets in a health care facility. The method collects tag data from readers which read data from automatic identification tags coupled to the assets; identifies a particular asset coupled to the tag data, and identifies the zone where the tag and thus the particular asset is located. The method normalizes the tag data from the readers into a standardized data format and checks for duplicate tag data based on certain configurable filters; analyzes the tag data with a set of pre-defined condition queries and sends appropriate action messages to an alert framework. Tag data, asset state information, zone information and other device information can be used in providing real time asset visibility. Based on this, the method provides a display for monitoring the assets and searching for the type of asset, location, image of the asset, and availability of the assets; and updates a central repository with changes in asset state information, and maintains a record of the assets for future utilization and for supporting a set of applications.

An example client system provides real time asset visibility and manages a plurality of assets. The system includes a plurality of radio frequency identification tags coupled to the plurality of assets for transmitting tag data; a plurality of active readers configured for receiving the tag data through a communication network; a location engine for identifying a particular asset and a zone where the particular asset is located from the tag data; a rules engine for analyzing the tag data through a set of pre-defined condition queries and for activating an alert framework which sends action messages to the appropriate departments in the health care facility; and a communication device including a display for displaying zone and other information of a particular asset to a set of users.

The client system further includes a device driver coupled to the plurality of various auto-identification devices (for example, active readers) for normalizing the tag data into a standardized data format; a tag manager which passes the tag data through a duplicate filter to check for duplicate tag data; a service bus for transmitting the tag data between the device driver, the tag manager, a location engine and a rules engine; an external communication module including a queue for publishing the changes in asset location, time outs of assets, and event rules, and including a web services client for receiving data from the exterior; and a central repository for storing the updated data from the web services client and for providing data to a set of external applications.

An example method provides automated identification of hospital staff who came in contact with a patient identified with a contagious disease. The method tags the patient with a hybrid radio frequency identification tag; tags hospital staff with individual infrared tags which include an infrared transmitter; sends a unique identification from the infrared transmitters to the hybrid radio frequency identification tags; detects when the hybrid radio frequency identification tag and the infrared transmitter are in close proximity; and sends data including a hospital staff ID and a patient ID to a service provider which analyzes the data to determine who among the hospital staff came in contact with the patient.

The invention also provides a set of applications including searching for availability and location of assets, data mining applications, dashboard applications, handheld applications, mapping applications, and reporting services.

Other aspects and example embodiments are provided in the Figures and the Detailed Description that follows.

### BRIEF DESCRIPTION OF THE DRAWINGS

FIG. 1 is a flow diagram illustrating steps in a method of providing real time asset visibility according to an embodiment of the invention;

FIG. 2 is a block diagram illustrating the client architecture for providing real time asset visibility according to an embodiment of the invention;

FIG. 3 is a block diagram illustrating the server architecture according to an embodiment of the invention;

FIG. 4-FIG. 15 are screen shots illustrating various functionalities of the Graphical User Interface (GUI) of the system according to an embodiment of the invention;

FIG. 16 is a flow diagram illustrating steps in a method of identifying the hospital staff who came in contact with a patient according to an embodiment of the invention;

FIG. 17 is a block diagram illustrating the RFID based voice call system according to an embodiment of the invention; and

FIG. 18 is a block diagram showing how types and attributes are associated with zones and assets, and how tags are used to track devices in the zones.

### DETAILED DESCRIPTION OF EMBODIMENTS

Embodiments of the invention teach a system and method for providing asset visibility in real time. Based on the real time asset visibility the invention also teaches, for an enterprise facility such as health care facility, a set of applications including searching for availability and location of assets, data mining applications, dashboard applications, handheld applications, mapping applications, and reporting services.

Embodiments of the invention automate workflows in an asset management system, especially in a hospital environment. A number of workflows start when a patient enters a hospital and continue until the patient is discharged. For example, if a patient moves from one room to another, the doctor needs to be notified. If a patient is about to enter a surgery room, a nurse needs to ensure that the equipment is clean. After surgeries, the billing department must be notified. A number of applications can be based on the associated asset information. For example, if a cleaning process is coupled to the asset, say a wheelchair, then when the patient leaves the room, the cleaning department can be notified by an automated message. The invention uses the real time location services and artificial intelligence to automate solutions to perform complicated equipment requisition and distribution, track asset requests, asset locations, automated workflows, reporting services, and mapping services. Users can access the system through their personal digital assistants (PDA), other hand-held devices, computers, laptops, etc.

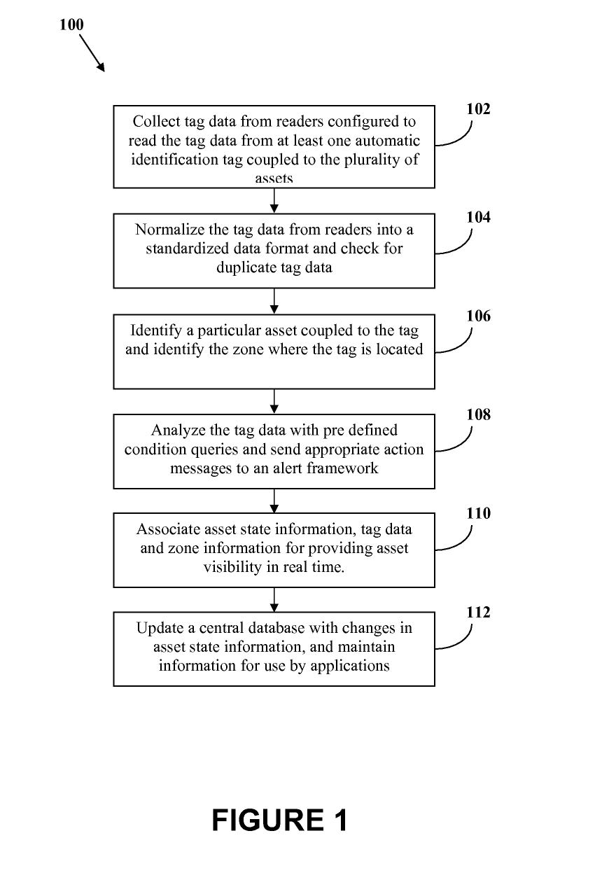

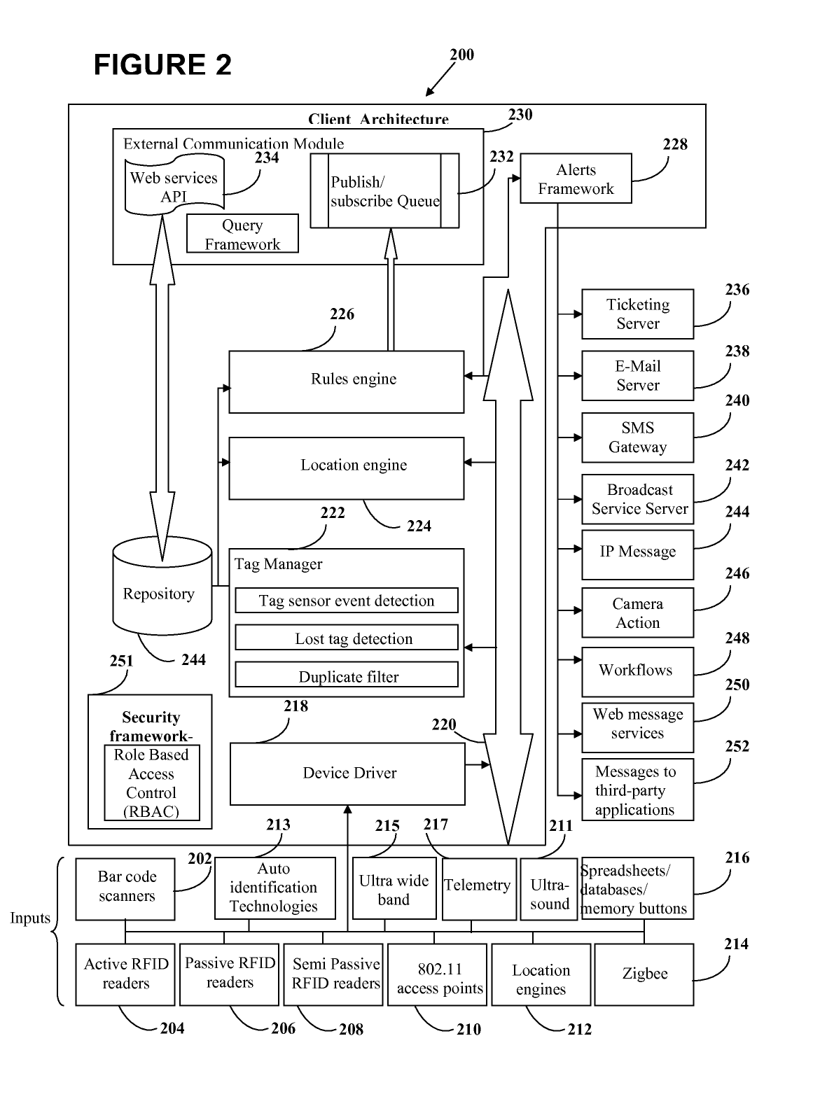

FIG. 1 is a flow diagram illustrating a method of providing real time asset visibility according to an embodiment 100 of the invention. Step 102 collects tag data from (FIG. 2) inputs 202-216 which are configured to receive tag data from the RFID tags coupled to the multiple assets. The tag data includes tag ID and tag state information. The inputs 202-216 include active RFID readers 204, passive RFID readers 206, semi-passive RFID readers 208, 802.11 access points 210, location engines 212, barcode scanners 202, Zigbee 214, and spreadsheets, databases and memory buttons 216, ultrasound 211, ultra wide band 215, Telemetry 217, and auto identification 213 technologies. Various ones of these inputs operate at different frequencies and have different ways of performing real time location. Hence, a step 104 normalizes the tag data from various readers into a standardized data format which includes Comma Separated Value (CSV) based and Extensible Markup Language (XML) based data formats.

Step 106 analyses the tag data and identifies and locates the zone of the particular asset coupled to the tag data. An asset can have multiple tags associated with it and a zone can have multiple devices associated with it. Devices include various auto identification technologies, sensor technologies and states. After the asset and zone have been identified from the tag data, a step 108 applies a pre-defined set of condition queries on the tag data. If the condition query result is true, an appropriate action message is sent to the alerts framework 228.

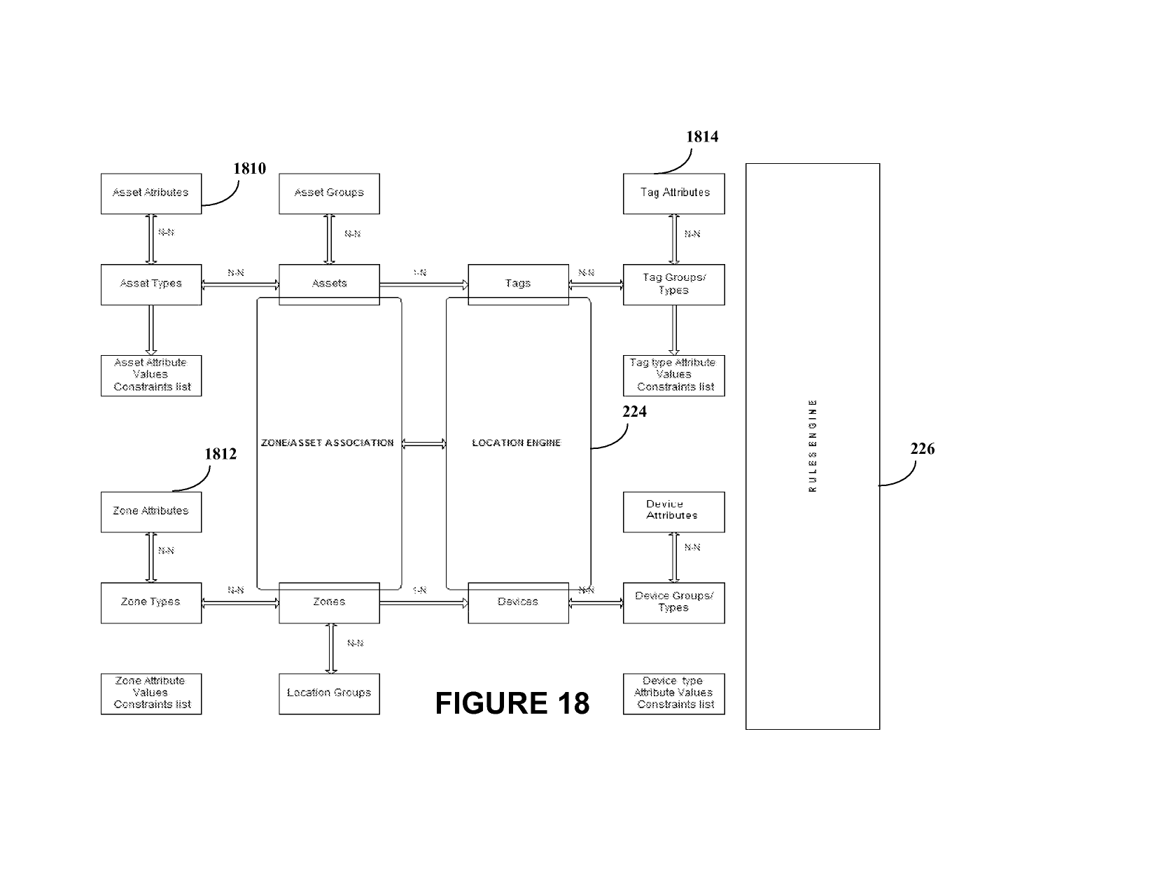

Step 110 associates the asset information (asset attributes 1810), zone information (zone attributes 1812) and tag data (tag attributes 1814) as shown in FIG. 18, which feeds a set of applications and enables providing asset visibility in real time. For example, in a hospital environment, a simple condition query whether an unclean IV pump is located in a new patient room would analyze the attributes (new) of the patient room (zone) and the attributes (unclean) of any IV pump (asset) located in the zone. If the condition is true, this generates a Short Message Service (SMS) and sends it as part of an action message through the alerts framework.

Step 112 updates a central repository or database with any changes in the asset state information and maintains the information for use by applications. The reliability of the asset and zone identification is preferably increased by combining existing information from the central repository, location information provided by the RFID readers, and state information of the assets.

FIG. 2 is a block diagram illustrating an embodiment of the client architecture 200 for providing asset visibility in real time. The client architecture includes multiple inputs (readers) 202-216, a device driver 218, a tag manager 222, an enterprise service bus 220, a location engine 224, a rules engine 226, an alerts framework 228, a web services Application Programming Interface (API) 234, and a publish/subscribe queue 232. The multiple inputs include barcode scanners 202, active RFID readers 204, passive RFID readers 206, semi-passive RFID readers 208, 802.11 access points 210, location point engines 212, Zigbee 214, and spreadsheets, databases and memory buttons 216, ultrasound 211, ultra wide band 215, Telemetry 217, and various auto identification technologies 213.

The tag data from the multiple inputs 202-217 is passed to the device driver 218 for that particular input (reader). The device driver 218 normalizes the tag data into a standardized data format and drives the normalized tag data onto the enterprise service bus (queue) 220. The tag manager 222 receives the tag data from service bus 220 and runs it through a duplicate filter whose criteria is set at system configuration and can be dynamically modified. Definitions of attributes and types can also be dynamically modified. If the tag is not a duplicate it is re-sent on the service bus 220 destined for location engine 224 or rules engine 226. The tag manager 222 runs a parallel process which checks for timed-out tag data and which sends the time-out records via the service bus 220 to location engine 224 or rules engine 226. The location engine algorithm associates the appropriate ‘asset’ and ‘zone’ with the augmented tag data (including details such as asset and zone attributes) as diagrammed in FIG. 18, and the augmented tag data is output on service bus 220.

The rules engine 226 passes the augmented tag data through ‘pre-defined condition queries.’ If a condition query result is true, engine 226 generates an appropriate action message and sends it to the alerts framework 228 which in response initiates an appropriate action externally including ticketing server 236 action, e-mail server 238 action, SMS gateway 240, broadcast service server 242 action, and others, such as IP message 244, camera action 246, activate workflows and deactivate workflows 248, web services messages 250, and messages to third party applications 252. The rules engine 226 publishes ‘change in asset location,’ ‘timeout of an asset after a certain period of time’ and rules events onto queue 232 in the external communication module 230. The location engine 224 takes environmental nuances into account and works in conjunction with the rules engine 226 to analyze anomalies and to provide an accurate and customized location, thus minimizing the likelihood of false alarms. A security framework 251 adds access privilege facility to the client architecture 200. Access privileges can be defined for the set of users based on their roles using a Role Based Access Control (RBAC) mechanism which has a multi-tenant architecture. Each cell (for example, each asset type in the asset types table. Similarly, zone types, locations, attributes etc) within the database can be defined as a privilege. Roles are defined as a collection of privileges with authorization to perform one or more of the following operations (view, update, delete) on each privilege. Roles can be hierarchical in nature. Each user can be assigned multiple roles within several organizations. A user can also be directly assigned or denied certain privileges (instead of roles). These directly assigned or denied privileges will override the privilege provided through the role that the user is assigned to. For example, a staff administrator who is authorized to service IV Pumps on the first floor of a hospital can have a role defined to permit access to only the first floor and to only view IV Pumps and update its attributes.

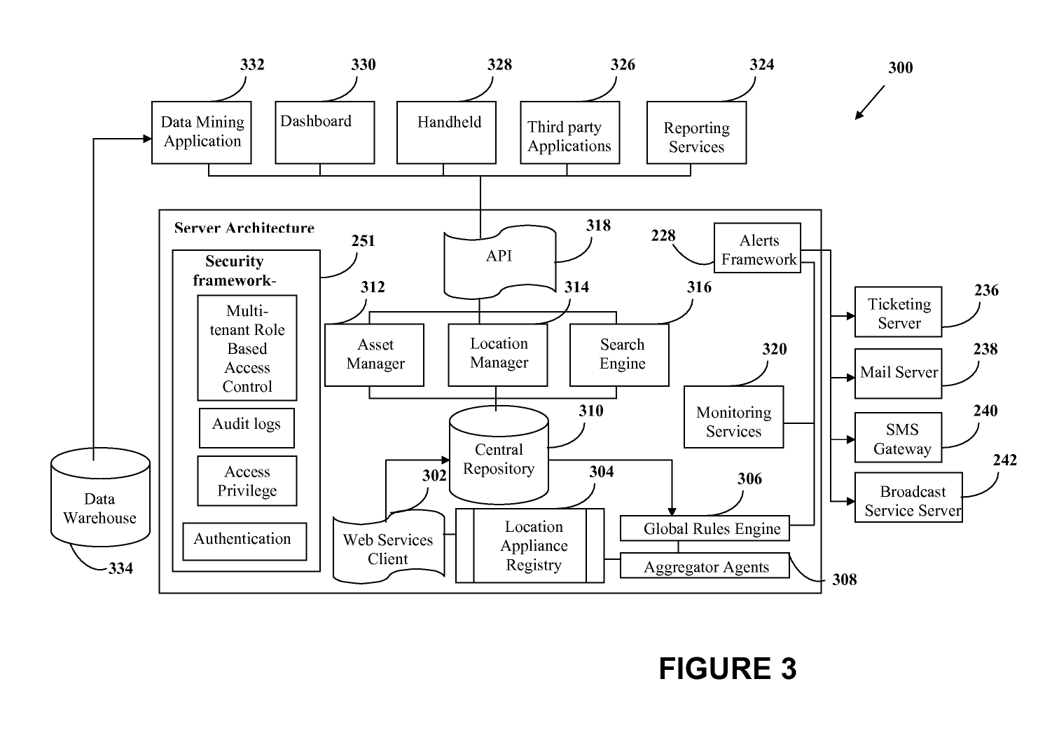

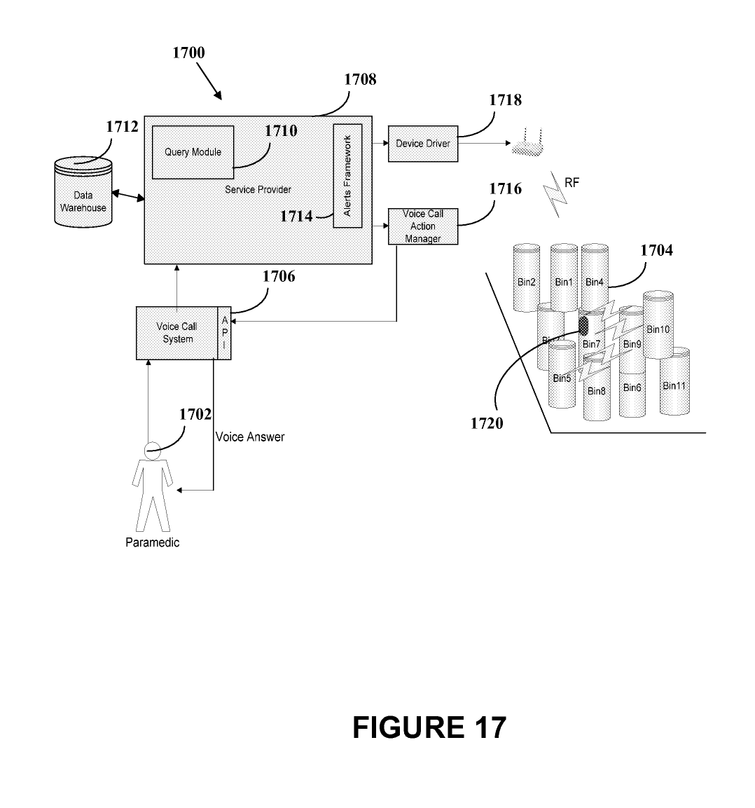

FIG. 3 is a block diagram illustrating a server architecture 300 according to the invention. The server architecture 300 includes a web services client 302 which communicates with web services API 234 in FIG. 2, location appliance registry 304, global rules engine 306, aggregator agents 308, a central repository 310, an asset manager 312, a location manager 314, search engine 316, a data warehouse 334 an API 318 for external applications such as data mining applications 332, dashboard applications 330, handheld applications 328, third party applications 326, and reporting services 324. FIG. 17 shows an example illustrating one of the reporting services of the invention. Server architecture 300 also includes an alerts framework 228 equivalent to alerts framework 228 in FIG. 2, a security framework 251 equivalent to security framework 251 in FIG. 2, and monitoring services 320.

The FIG. 2 queue 232 information is received by the web services client 302. The central repository 310 is updated with the data (asset, location, rules) received from the client. The asset, location, rules and state information is available to various front end applications, e.g., data mining applications 332, dashboard applications 330, handheld applications 328, third party applications 326, and reporting services 324. The asset, location, rules, and state information is also passed through the global rules engine 306 which executes system-wide rules. The security framework 251 maintains an account of access actions in audit logs, defines the access privileges, and performs authentication for securing system logins.

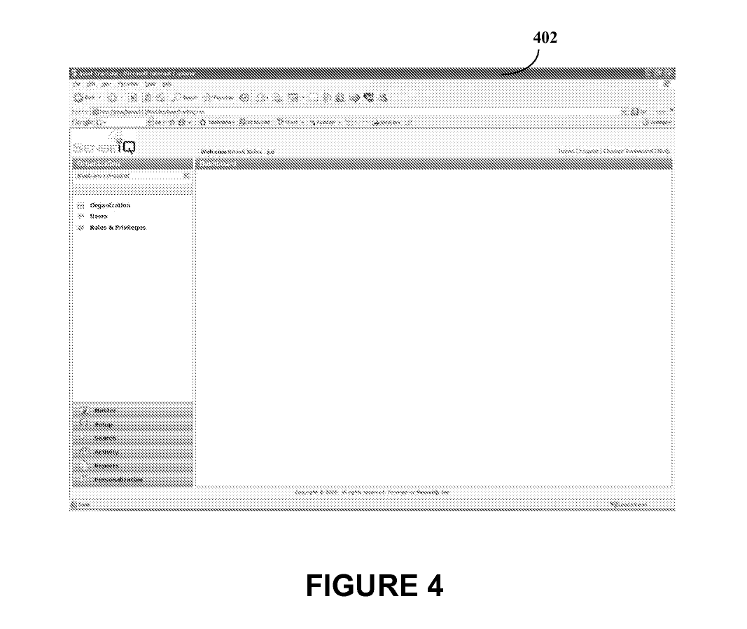

FIG. 4-FIG. 15 are screen shots illustrating various applications or functionalities of the Graphical User Interface (GUI) 402-1502 according to an embodiment of the invention.

At the first screen or “main menu” (not shown) the system login is secured with user authentication, audit logs and access privileges. An administrator (user) has to log in to the system using a user ID and password. The main menu includes buttons for master, setup, search, activity, reports and personalization.

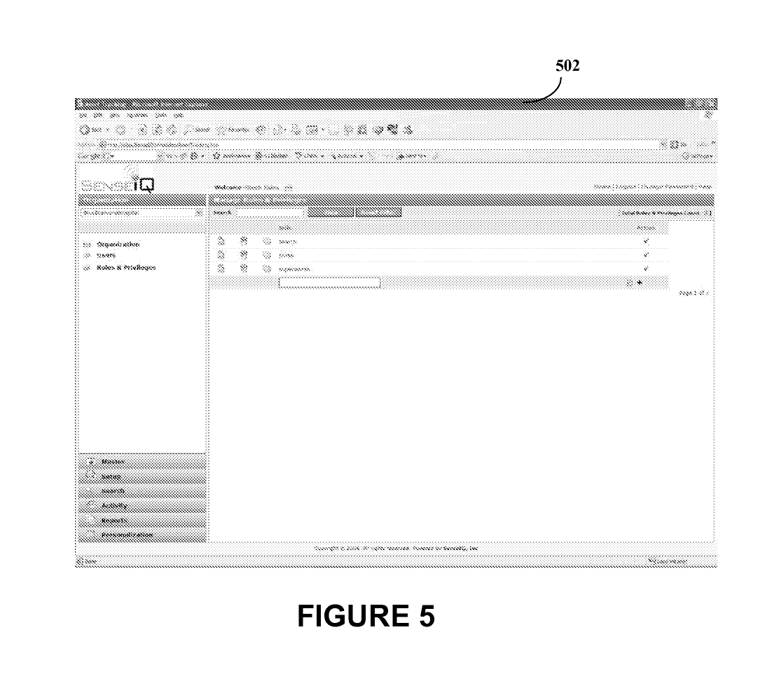

FIG. 4 illustrates the master menu 402 where a user can manage organization, users and roles and privileges. Under “organization” the user can select an existing organization to edit or access asset visibility in real time, or add a new organization. Under “users” the user can manage the list of users, view and edit existing users, or add new users. FIG. 5 shows how under “roles and privileges” certain user groups can be managed. For example, a nurse's role can be defined to have access to only certain facilities.

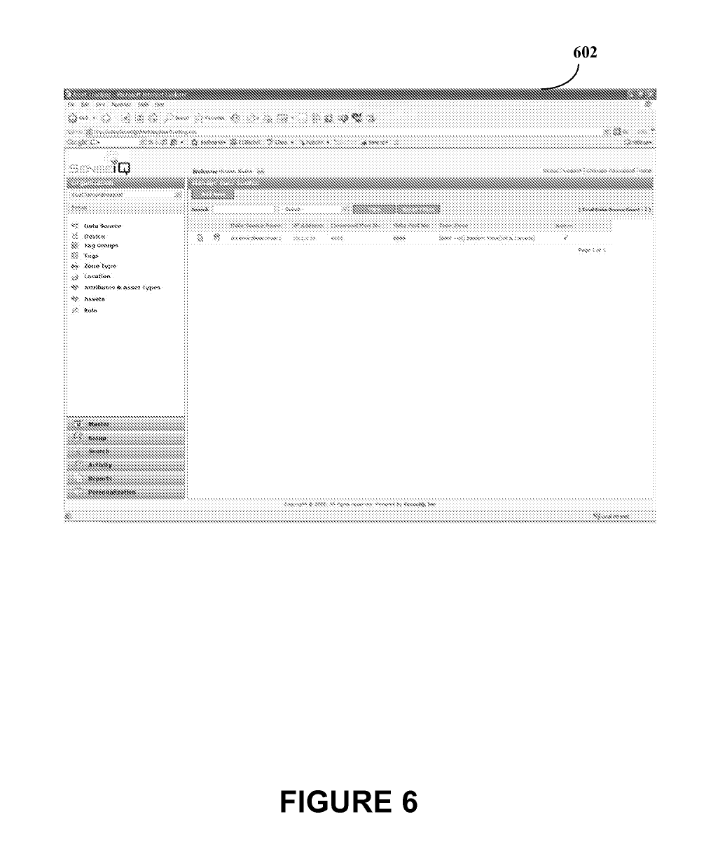

In the setup menu, the user can manage data sources, devices, tag groups, tags, zone types, locations, attributes and asset types, assets, and rules as illustrated in FIG. 6-FIG. 12.

FIG. 6, 602 illustrates the example of managing data source inner wireless Driver1. The IP address, command port number, data port number, time zone and activity state of the data source are shown on the GUI. Users can edit this data source, perform a test connection and update this information. The devices can also be managed and device data can be updated including the device type, purchase date and installation date. A description of the device is also available.

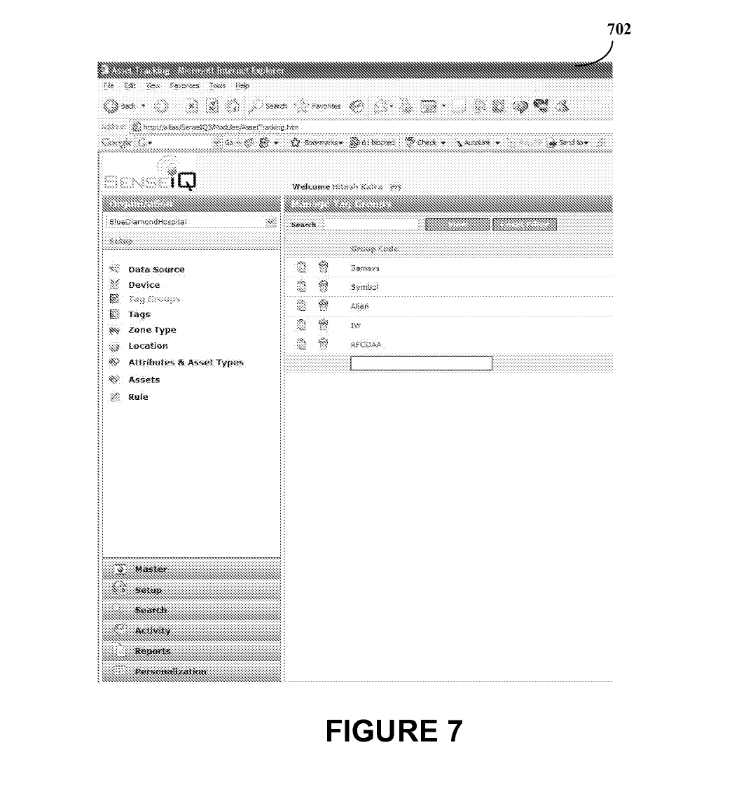

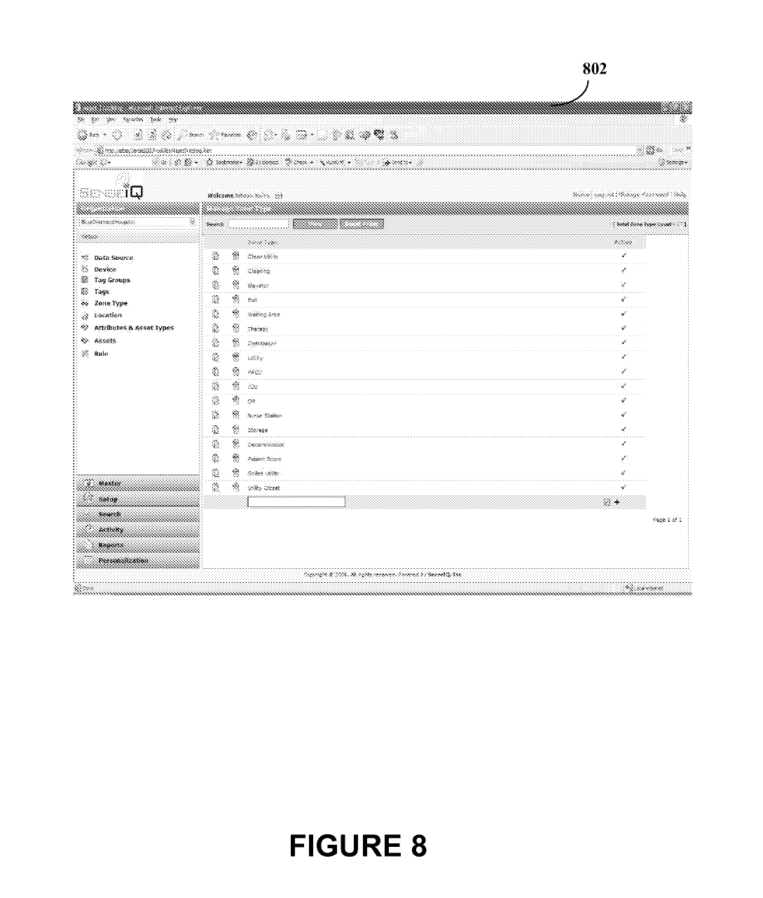

FIG. 7 illustrates the manage tag groups screen 702. Different types of RFID tags coupled to the assets can be sorted into type groups and given group codes. FIG. 8 shows the manage zone type screen 802. Examples of different zone types in a hospital environment include clean utility, elevator, exit, waiting area, distribution area, lobby, operation theatre, patient room, etc. Users can view the activity statuses of the different zones. The user can also manage different asset types, and each of these asset types can have different data types (e.g. Strings). Different types of assets are grouped into a composite asset group, which is a collection of a number of assets of different asset types grouped into one category. Rules are defined in the rules engine to track groups of assets in the composite asset group. If there is any mismatch while tracking the assets, the alert framework generates an action message. For example, in a hospital environment, when a bin with defined asset types leaves the door, a self check function can be performed to confirm whether the bin contains ‘x’ number of assets of each of ‘y’ number of asset types. This composite asset type functionality can also be used in real-time inventory management systems.

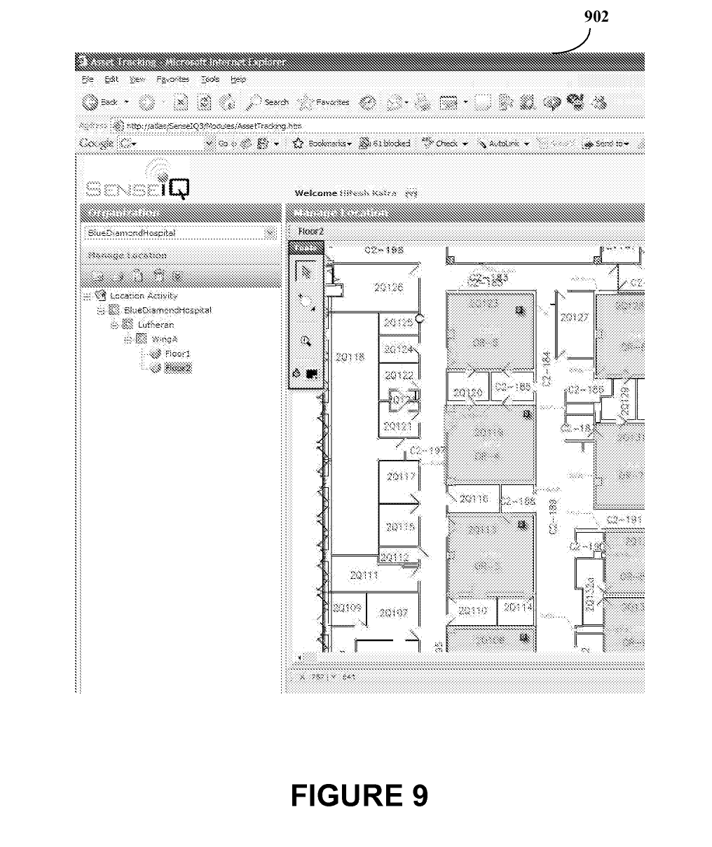

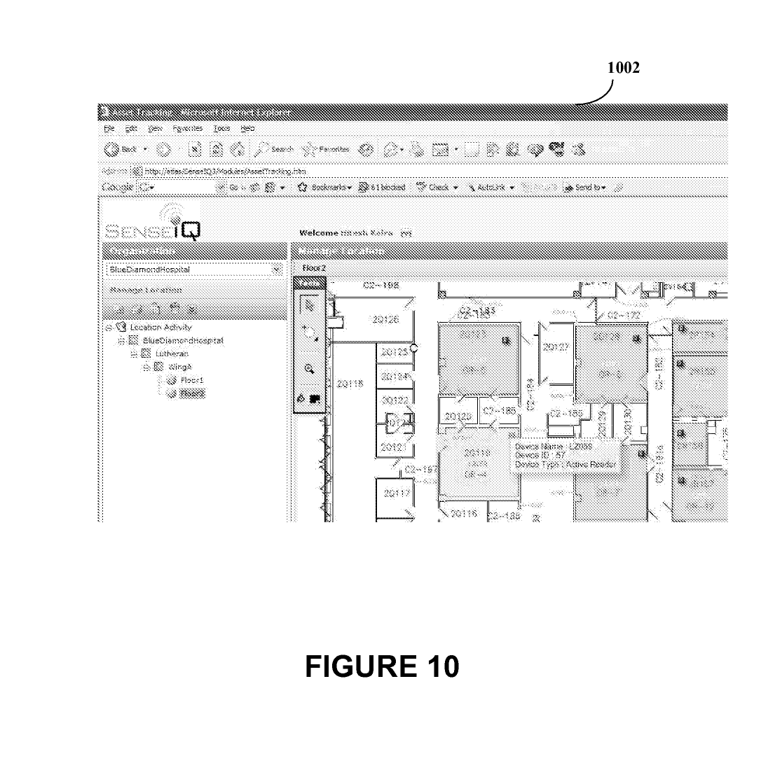

FIG. 9 illustrates a screen 902 for managing location activities. A user can view a geographical map of the location and can view the location of any asset of interest. The system provides a zone-level view of assets and their positions. As shown in FIG. 10, a user can highlight a particular asset and view activity details including device name, device number and device type. This is important in reducing operating costs and time spent searching for assets. For example, a doctor wanting to know where a surgery will be performed can view the map and get updated information about operation theatres in a particular section. The system of the invention further maintains a record of the current and previous states of users, and based on an intelligence algorithm, displays the frequently visited areas and related information on the geographical map.

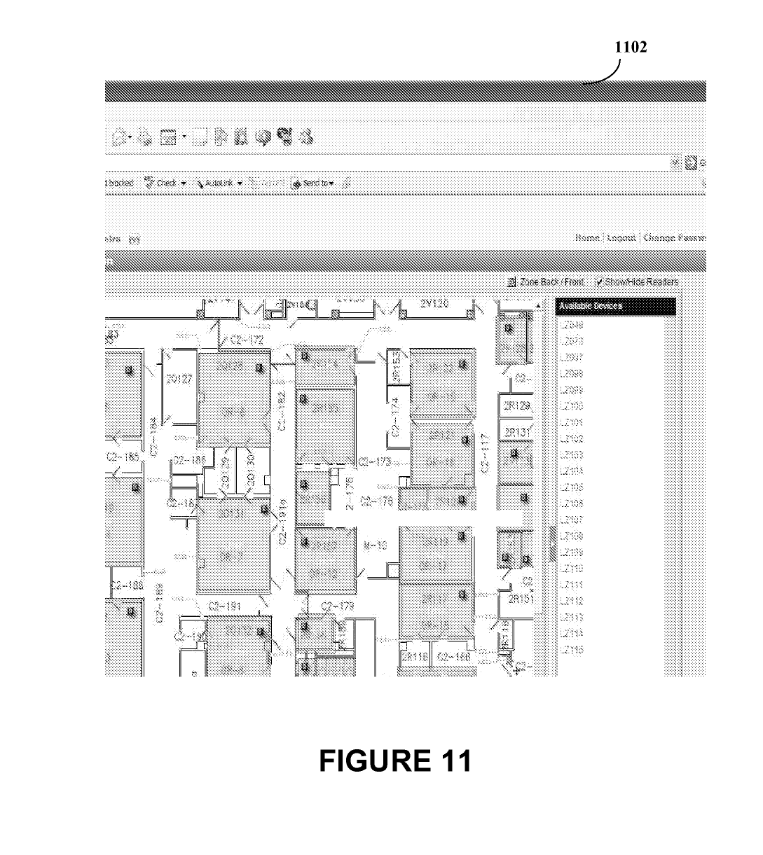

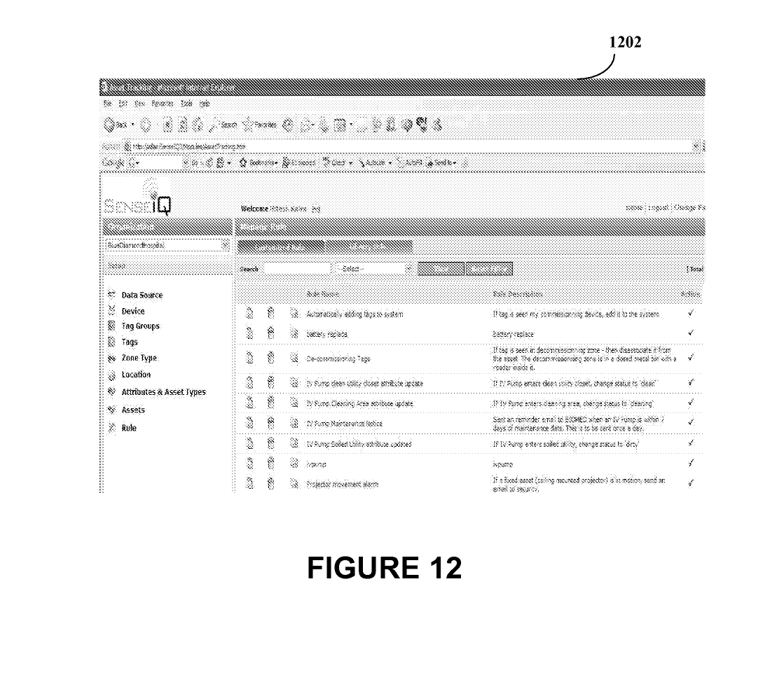

FIG. 11 shows the number of devices available in a selected zone of the hospital. For example, a surgery assistant can use this locating facility to determine which equipment is clean and available before surgery. From the setup screen, the user can navigate to a manage rules screen. The rules are divided into customized rules and library rules. FIG. 12 shows a screen 1202 of customized rules, which can be dynamically modified according to requirements. A rule named ‘IV pump cleaning area attribute update’ changes the status of an IV pump to ‘clean’ if it enters the cleaning area. When an IV pump is within seven days of its scheduled maintenance, a rule named ‘IV pump maintenance notice’ sends a reminder e-mail once a day to the biomedical department. A ‘projector movements’ rule could be “if ceiling mounted projector is in motion, send an e-mail to the security department.”

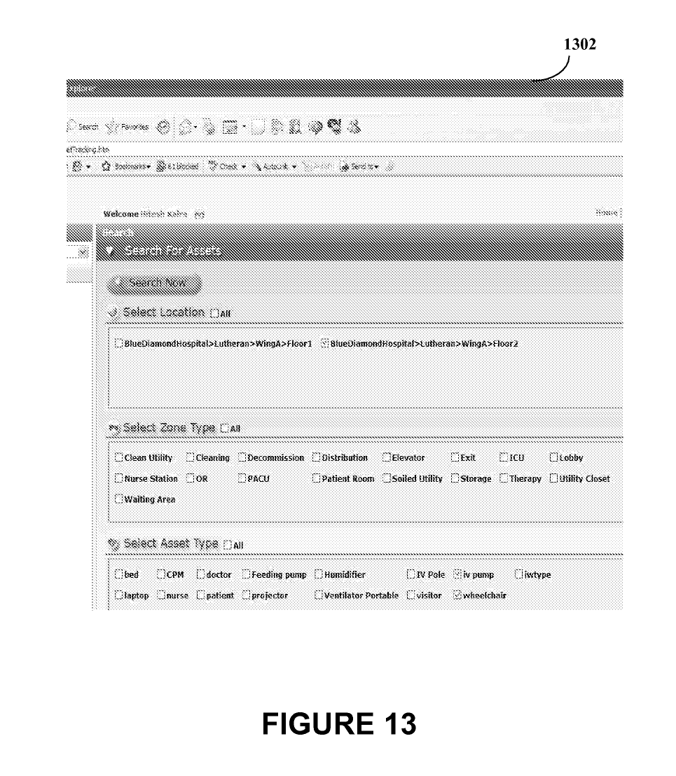

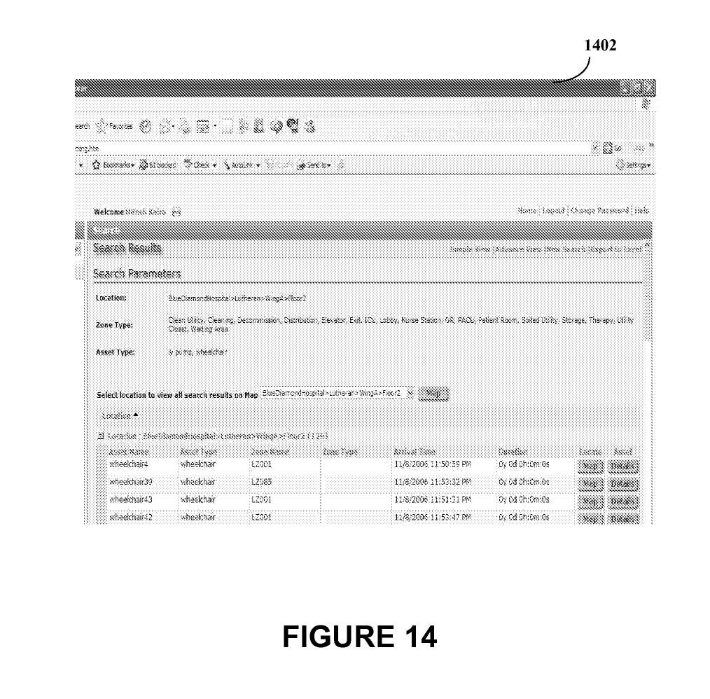

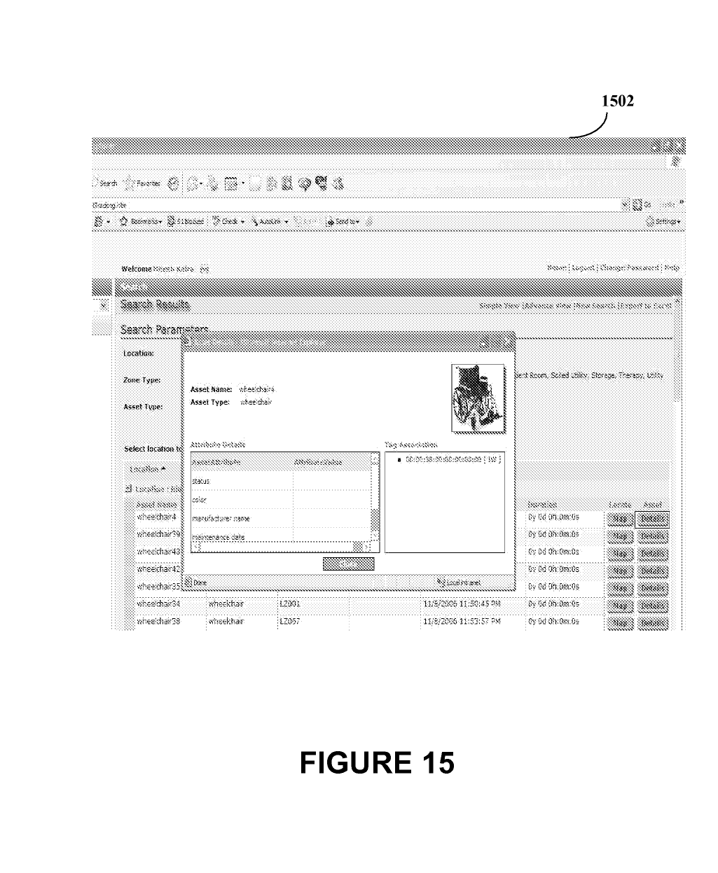

FIG. 13-FIG. 15 are screen shots illustrating the search facility of the system according to an embodiment of the invention. FIG. 13 screen 1302 shows search areas including location search, zone type search and asset type search. FIG. 14 screen 1402 shows how, if the asset name and location are already known, more advanced searching is possible. The search provides asset name, asset type, zone name, zone type, arrival time and duration. For example, if the user searches for a wheelchair, the findings indicate the number of wheelchairs available, wheelchair 4 (asset name), wheelchair (asset type), LZ 001 (zone name), and time of arrival. The user has the advantage of locating a particular asset in the map and views the asset details. The user can also separately view the image of the asset along with the asset details as shown in FIG. 15, 1502. The system provides indoor and outdoor asset tracking facility. In the global view, the system's location algorithm automatically uses telemetry systems such as Global Positioning System (GPS) technology to track assets, but as the user zooms into the map view to narrow the vision/area of coverage, the system automatically and seamlessly switches to the indoor positioning system.

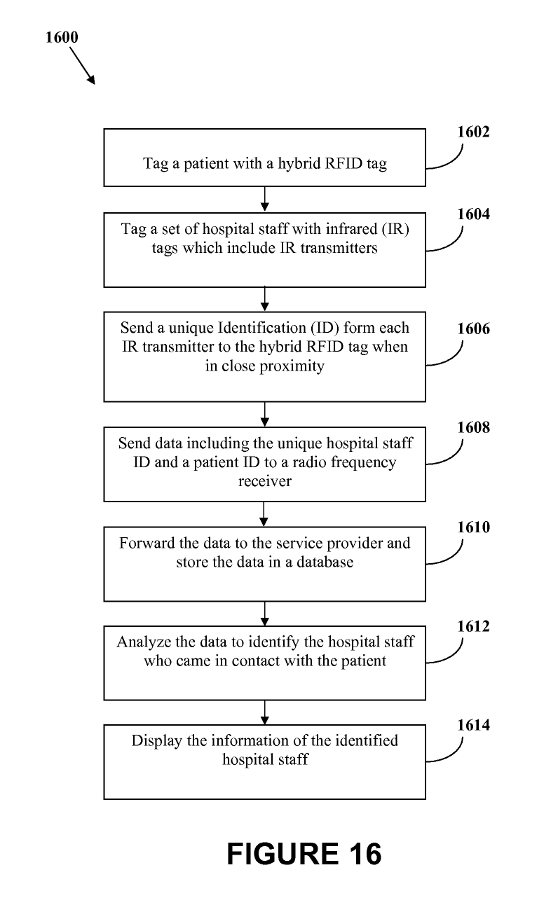

FIG. 16 is a flow diagram illustrating steps in a method 1600 according to an embodiment for identifying the hospital doctors, nurses, clinicians and other staff who have come in contact with a patient who was diagnosed with a contagious disease (for example, avian flu). Step 1602 tags the patient with a hybrid Radio Frequency ID (RFID) tag including a Radio Frequency (RF) transmitter and an Infrared (IR) receiver. Step 1604 tags the hospital staff with IR tags which include IR transmitters. Step 1606 sends a unique staff ID from the IR transmitters on the staffers to the hybrid RFID tags on the patients when IR transmitter and hybrid RFID tags are in close proximity (e.g., 2 meters). Step 1608 sends the data including the unique hospital staff ID and patient ID via communication mechanism such as RF to one of several receivers placed throughout the hospital environment.

In step 1610 the RF receiver forwards the data to the service provider and stores the data in the database 244. Step 1612 analyzes the data to identify the hospital staff who came in close proximity with the patient. Step 1614 displays the information of the identified hospital staff. This information can be displayed in the form of an Excel spreadsheet, a chart view or a map view to trace the location of the patient and hospital staff within a certain timeframe.

FIG. 17 is a block diagram illustrating an RFID based voice call system 1700 according to an embodiment of the invention. For example, a paramedic (user, 1702) may be searching for a portable electrocardiogram (EKG) machine. The user 1702 places in the voice call system 1706 a voice query for an as-yet unidentified bin which contains a portable EKG machine. The voice call system 1706 receives the query and sends it through the web services Application Program Interface (API) to the service provider system 1708. The query module 1710 queries the data warehouse 1712 for the bin ID and the tag ID of a bin containing a portable EKG machine which is available for usage. The data warehouse 1712 sends its response, bin 1720, to the alerts framework 1714 (equivalent to alerts framework 228 in FIG. 2).

The alerts framework 1714 forwards the bin ID response through the voice call action manager 1716 to the voice call system 1706. The alerts framework 1714 also initiates an action through the device driver 1718 to activate the RFID tag mounted on the identified bin 1720. The voice call system 1706 receives the bin ID and announces to the user 1702 that bin number 1720 includes the available portable EKG machine. Simultaneously, a Light Emitting Diode (LED) and/or a sound buzzer is activated on the particular bin 1720, thus enabling the user 1702 to visually and/or audibly locate it.

The forgoing description sets forth numerous specific details to convey a thorough understanding of the invention. However, it will be apparent to one skilled in the art that the invention may be practiced without these specific details. Well-known features are sometimes not described in detail in order to avoid obscuring the invention. Other variations and embodiments are possible in light of above teachings, and it is thus intended that the scope of invention not be limited by this Detailed Description, but only by the following Claims.

I claim:

1. A method for providing real time asset visibility and management of a plurality of assets comprising the steps of:
collecting tag data from a plurality of automatic identification technology detectors configured to read said tag data from at least one radio frequency identification tag coupled to said plurality of assets, wherein said automatic identification technology detectors include radio frequency readers;
identifying a particular asset coupled to the tag data and identifying a zone where said particular asset is located; and
associating asset state information, tag data information, zone information and other device information in the environment of the asset.

2. The method of claim 1, further comprising:
normalizing the tag data from the plurality of active readers into a standardized data format;
checking for duplicate tag data based on configurable filters;
analyzing the tag data with pre-defined condition queries
sending appropriate action messages to an alerts framework;
providing a display for monitoring the assets;
searching for the type, image, location, and availability information of the assets; and
updating a central repository with a change in said information for future utilization and for supporting associated asset management applications.

3. The method of claim 2 wherein the alerts framework comprises a ticketing server, a mail server, a short service message gateway or a broadcast services server.

4. The method of claim 2 wherein said action messages comprise short service messages, electronic mail, internet protocol messages, camera actions, ticketing server actions, broadcast actions, activating workflows, deactivating workflows, web services messages, or messages to third party applications.

5. The method of claim 2 wherein said associated applications comprise data mining applications, dashboard applications, handheld applications, mapping applications, reporting services or other third party applications.

6. The method of claim 5 wherein
said data mining applications include
searching for a particular asset and viewing the status depending on the user interest,
viewing a maintenance record of the asset, and
providing requesting facility for accessing a particular asset available;
said reporting services include
generating automated messages to the respective departments,
posting a performed action in a health care facility using artificial intelligence, and
notifying the respective departments; and
said mapping applications include allowing a user to view
the distribution of assets, and
details including maintenance records of a particular asset.

7. The method of claim 1 wherein said device information comprise auto identification technologies, sensor technologies and states.

8. The method of claim 1 wherein the plurality of assets comprise movable assets.

9. The method of claim 1 wherein the plurality of assets comprise stationary assets.

10. The method of claim 1 wherein the plurality of assets comprise a composite asset group.

11. The method of claim 1, wherein access controls are provided to restrict user's access to the system, zones and assets based on the privileges granted by a role based access control system.

12. A system for providing real time asset visibility and management of a plurality of assets comprising:
a plurality of radio frequency identification tags coupled to said plurality of assets for transmitting tag data;
a plurality of detectors configured for receiving said tag data through a communication network;
a location engine for identifying a particular asset and a zone where said particular asset is located from the tag data;
a rules engine for analyzing the tag data through a set of pre-defined condition queries and for activating an alerts framework; and
a display for displaying asset information and zone information of the particular asset to a user.

13. The system as in claim 12 further comprising:
a device driver coupled to said plurality of active readers for normalizing the tag data into a standardized data format;
a tag manager which passes the tag data through a duplicate filter to check for duplicate tag data based on configurable filters;
an enterprise service bus for transmitting the tag data between said device driver, said tag manager, said location engine and said rules engine;
an external communication module for publishing, in a queue, changes in asset location, time-out of an asset after a certain period of time, and event rules;
a web services client for receiving data from the external communication module;
a central repository for storing updated data from said web services client and for providing data to a set of applications associated with the system; and
a global rules engine for executing a set of system wide rules.

14. The system of claim 12 wherein the location engine and the rules engine are used together to provide asset tracking in a enterprise facility such as health care facility.

15. The system of claim 12, wherein said plurality of automatic identification readers include active radio frequency identification readers, passive radio frequency identification readers, semi-passive radio frequency identification readers, location engines, bar code scanners, spread sheets, data bases or memory buttons.

16. The system of claim 12, wherein the alerts framework is activated by sending a set of action messages generated from said rules engine after analyzing the tag data.

17. The system of claim 16, wherein the alerts framework comprises a ticketing server, a mail server, a short service message gateway or a broadcast services server.

18. The system of claim 16, wherein said action messages comprise short service message, electronic mail, internet protocol message, camera action, ticketing server action, broadcast action, activating workflows, deactivating workflows, web services message or messages to third party applications.

19. The system of claim 13, wherein said associated applications comprise data mining applications, dashboard applications, handheld applications, mapping applications, reporting services or other third party applications.

20. The method of claim 12 wherein the plurality of assets comprise movable assets.

21. The method of claim 12 wherein the plurality of assets comprise stationary assets.

22. The method of claim 12 wherein the plurality of assets comprise a composite asset group.

23. A method of determining which staff members of enterprise facility such as a health facility have encountered a given patient, comprising:
tagging patients each with a hybrid RFID tag having a unique patient ID number;
tagging hospital staff members each with an IR tag that continuously transmits a unique staff ID;
detecting, by the hybrid RFID tags, the unique staff ID when a tagged staff member has an encounter within close proximity of a tagged patient;
sending, by the hybrid RFID tag, the unique staff ID and the unique patient ID encounter information to one or more RF receivers positioned in the facility;
forwarding, by the RF receiver, this information to a tracking service provider; and
analyzing and storing the information on encounters in specific zones of the facility.

24. The method of claim 23 further comprising: displaying information of the identified hospital staff who came in contact with the patient.

25. The method of claim 23 wherein the automatic identification tag includes a radio frequency transmitter and an infrared receiver.

26. A computer program product for providing real time asset visibility and management of a plurality of assets, said computer program product including instructions for causing a computer system to perform the steps of:
collecting tag data from a plurality of active readers configured to read said tag data from at least one active radio frequency identification tag coupled to said plurality of assets;
identifying a particular asset coupled to the tag data and identifying a zone where said particular tag is located; and
associating asset state information, tag data, zone information and other device information in the environment of the asset.

27. The computer program product of claim 26 further including instructions for causing the computer system to perform the steps of:
normalizing the tag data from the plurality of active readers into a standardized data format;
checking for duplicate tag data based on configurable filters;
analyzing the tag data with pre-defined condition queries;
sending appropriate action messages to an alerts framework;
providing a display for monitoring the assets;
searching for the type of asset, location, image of the asset, and availability of the assets; and
updating a central repository with a change in said asset state information and maintaining a record of the assets for future utilization and for providing a set of applications.
